(19) United States
(12) Patent Application Publication
(10) Pub. No.: US 2022/0061686 A1
(43) Pub. Date: Mar. 3, 2022

(54) HEART RATE VARIABILITY NOTIFICATION DEVICE

(71) Applicant: Harry Zeritis, Scarborough (CA)

(72) Inventor: Harry Zeritis, Scarborough (CA)

(21) Appl. No.: 17/006,971

(22) Filed: Aug. 31, 2020

(51) Int. Cl. A61B 5/024; A61B 5/0245; A61B 5/0255; A61B 5/0408; A61B 5/00; A61B 5/044; A61B 5/04

(52) U.S. Cl. CPC A61B 5/02405; A61B 5/0245; A61B 5/0255; A61B 5/04085; A61B 5/6824; A61B 5/044; A61B 2562/0209; A61B 5/7455; A61B 5/746; A61B 5/486; A61B 5/02438; A61B 5/04012; A61B 5/7405

(57) ABSTRACT

The invention and embodiments thereof as described herein relate to a wearable device which can monitor HRV (heart rate variability) of the wearer. The device is programmed to warn the wearer if their HRV is indicative of them entering into a subconscious negative state of mind or a situation that is causing them higher stress level. Predetermined HRV rhythms will be used to determine if the wearer is in a negative state, or experiencing a higher than normal stress level.

### FIELD OF THE INVENTION

The present disclosure relates to, a wearable device particularly to a wearable device which can monitor the heart rate variability (HRV) of the wearer and more particularly to a wearable device that can warn the wearer when their HRV is indicative of a negative state of mind or higher stress level.

BACKGROUND

Each individual has a heart that beats during any given moment at a certain rate generally measured in beats per minute. The heart rate of each individual, however, is generally not constant. Instead, an individual's heart rate varies typically from beat to beat. The variation in heart rate is referred to as Heart Rate Variability (HRV). HRV depends upon moment to moment influences of sympathetic and parasympathetic activity of an individual's body. HRV is integral to an individual's response to a wide range of daily influences, including those due to external environment, and those factors directly associated with an individual including breathing patterns and internal physiology.

An individual's ability to adapt to these influences is directly related to HRV. Thus, HRV can be an important indicator of an individual's fitness level, health or mental condition. For instance, impairment of an individual's adaptive abilities may lead to development of cardiac, respiratory, immune or stress related disorders.

Many disorders typically result in decreased HRV of the individual. Decreased HRV relates to many disorders including hypertension, arrhythmia, myocardial infarction, chronic fatigue, panic attacks and stress related disorders. The decrease in HRV in these and other disorders typically is related to a decrease in parasympathetic activity, an increase in sympathetic activity, or a disruption in how these two systems interact with one another.

Many health, fitness and wellness conditions could be improved if HRV of an individual was known by the individual throughout the day. Feedback given to an individual based on proper monitoring of HRV could then prompt some type of immediate corrective or therapeutic action by the individual. For instance, corrective or therapeutic action could be adopted during events causing higher than normal stress levels, chronic fatigue, panic disorders, irregular respiration and falling into a negative state of mind.

Unfortunately, prior art devices which monitor HRV are not programmed to provide timely and simple feedback to individuals wearing the devices without user input or effort. Other prior art devices have been portable, however, these devices determine only heart rate levels, while others that track HRV do not provide that prompt feedback that can be used in any situation to stay positive as the wearer is going about their day to day chores. The lack of feedback of prior art monitors and the lack of HRV monitoring of other prior art devices that do not alert individuals of HRV conditions during their normal activities throughout the day and night, results in an unfulfilled need for devices to improve health and fitness conditions of individuals.

### BRIEF SUMMARY

It is an object of the invention to provide a heart rate variability notification device.

In accordance with an aspect of the invention there is provided a heart rate variability notification device for a user, the device comprising: an output system configured to transmit a sensory signal perceptible by the user based on the output system receiving an activation signal; a plurality of sensors configured to produce electrical signals based on electrical activity of the user's heart; a housing sized and configured to be worn by the user; a memory housed by the housing wherein the memory is configured to store heart rate variability notification criteria; and a processor housed by the housing and coupled to the plurality of sensors and the output system and the memory, the processor configured to receive digital signals associated with the electrical signals produced by the sensors, the processor configured to measure and record heart rate variability of the user based on the digital signals and to transmit the activation signal to the output device based on whether the measured heart rate variability satisfies the heart rate variability feedback criteria, the processor configured to modify the heart rate variability feedback criteria based on the measured heart rate variability.

The advantages and features of the present invention will become better understood with reference to the following more detailed description and claims taken in conjunction with the accompanying drawings in which like elements are identified with like symbols.

To easily identify the discussion of any particular element or act, the most significant digit or digits in a reference number refer to the figure number in which that element is first introduced.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an item 100 in accordance with one embodiment of the invention.

FIG. 2 illustrates an aspect 200 of the subject matter in accordance with one embodiment.

FIG. 3 illustrates an aspect 300 of the subject matter in accordance with one embodiment.

FIG. 4 illustrates an aspect 400 of the subject matter in accordance with one embodiment.

FIG. 5 illustrates an aspect 500 of the subject matter in accordance with one embodiment.

FIG. 6 illustrates an aspect 600 of the subject matter in accordance with one embodiment.

FIG. 7 illustrates, in graphical format, results of HRV measured over a 120 second timeframe using an embodiment of the invention.

### DETAILED DESCRIPTION

The present invention relates to physiological monitoring devices and, more particularly, to a heart rate variability notification device and monitoring system.

The invention overcomes the limitations of the prior art and provides additional benefits by providing a heart rate variability (HRV) feedback monitor system. The HRV feedback monitor allows for expanded accessibility under a wide range of activities. As part of the feedback provided, the HRV feedback monitor furnishes effective feedback that is directly related to areas of concern. The feedback is also discrete in nature. This in addition to other aspects of the invention provides effective, discrete, and timely HRV monitoring and feedback without being overly burdensome. Thus, the invention overcomes the problems and difficulties posed by the prior art systems and provides numerous additional benefits.

A feedback monitor, and in particular, a device and corresponding method for a heart rate variability (HRV) feedback monitor system for monitoring heart rate variability of a user's body is described in detail below. In the following description, numerous specific details are provided, such as specific configuration of the apparatus, circuit components, ways of wearing the HRV feedback monitor, HRV criteria used for feedback, etc., to provide a thorough understanding of the embodiments of the invention. One skilled in the relevant art, however, will recognize that the invention can be practiced without one or more of the specific details or with other processes, configurations, hardware, etc. In other instances, well-known structures or operations are not shown or described in detail to avoid obscuring the description of the embodiments.

Each of the circuits whose function and interconnection are described in connection with the figures is of a type known in the art, and one skilled in the art would be able to use such circuits in the described combination to practice the present invention. The internal details of these particular circuits are not part of, nor critical to, the invention. Therefore, a detailed description of the internal circuit operation is not required. Similarly, the specific formulae and algorithms used in in the calculation of the HRV are not part of, nor critical to, the invention and a worker skilled in the field would be capable of programming the device to notify the wearer of a problematic HRV whether it be clinical or psychological.

The depicted embodiment of the invention solves problems of prior art devices that do not provide associated feedback based on HRV criteria. The depicted embodiments are lightweight and compact, along with other characteristics, so can be worn throughout the day. Also, the depicted embodiments are simple to operate, which promotes ease of use. Further, the depicted embodiments have a feedback mechanism which is discrete allowing use of the HRV feedback monitor in most situations and environments common to everyday life. The feedback is automatic and effortless for the wearer. Therefore this is the only embodiment that will enable the wearer to stay positive throughout the day with discrete immediate warnings. This dramatically increases the amount of positive feedback related to modulation of HRV resulting in improvement in their mental health.

The present invention relates to devices for real-time monitoring of heart rate variability (HRV), more particularly to HRV monitoring systems and devices that are adapted to give immediate feedback to the subject concerning their current physiological condition and any pertinent changes in their physiology. The means of notification may be haptic, auditory or visual.

Heart Rate Variability (HRV) has been widely used as a scientific measurement for monitoring the physiology of both human and animal subjects. HRV is the physiological characteristic of the variation in timing between heartbeats. The heartbeat originates in specialized tissue in the heart called the sino-atrial (SA), continuously generating an electrical impulse that spreads throughout the heart muscle. This initiates process of heart muscle contraction, a well-synchronized pump that sequentially constricts all 4 chambers of the heart (two atria and two ventricles). The SA node signals (approximately 100-120 impulses per minute when the heart is at rest) are regulated by the autonomic nervous system (ANS) by inhibiting some of the electrical impulses. The net effect results in a normal resting heart rate (in healthy individuals) of about 55 to 70 beats per minute (at rest).

This autonomic nervous system is that part of the nervous system that is not under conscious control. It controls the organs and systems of the body that are rhythmic, regular and automatic such as breathing, digestion and heart rate. There are two branches of the autonomic nervous system: sympathetic and parasympathetic. The sympathetic nervous system provides the basal heartbeat (HB) rhythm based on overall need. This response of the heart rate to normally encountered levels of sympathetic stimulation is modulated by parasympathetic stimulation. This heartbeat response to the parasympathetic nervous system, in contrast to the sympathetic nervous system, occurs rapidly and frequently. The deceleration of the heartbeat is almost instantaneous. It only takes 1 or 2 heartbeats to see these changes take place, slowing the heart rate.

HRV analysis can be used in both clinical and non-clinical applications for a diverse range of evaluations. In healthy individuals, the HR is variable. It fluctuates and generally, greater variability (or HRV) correlates with better health. Higher HRV indicates a healthy autonomic nervous system, and in particular, healthy balance between the sympathetic and parasympathetic systems. A decreased HRV is an early, accurate indicator that the autonomic nervous system is out of balance. The lower the HRV, the greater the imbalance in autonomic control and the greater the likelihood of poor health, both now and in the future.

Clinical applications for HRV analysis are related to cardiac health, and are indications that are shown to directly relate to health changes with many chronic and critical health conditions. Included are, but not limited to, risk of a cardiac event, occurrence of diabetes, episodic and chronic mental health conditions, sleep apnea, SIDS, exposure to and incidence of allergic reactions. In non-clinical applications, it has been shown that HRV is effective in indicating a variety of physiological conditions, including higher than normal stress levels, chronic fatigue, panic disorders, irregular respiration and falling into a negative state of mind. During vigorous exercise, HRV has been shown to be a marker for entering lactate threshold or anaerobic metabolism. Further, it is shown to be an indicator of physical fatigue, exercise capacity, endurance, and overall fitness.

There are several ways to measure and analyze HRV. Photoplethysmography (PPG) is a non-invasive and simple to use technique that can conveniently access capillaries in certain areas of the body, for example fingertip, earlobe or wrist or ankle. Using differential light absorption characteristics and an optical sensor, PPG detects changes in the pulse waves generated by blood flow through the microcirculation. In this way an accurate estimate of HRV can be obtained. The present invention features system, devices, and methods for real-time HRV monitoring. The HRV systems and devices of the present invention are adapted to give immediate feedback to the subject concerning their current physiological condition and any pertinent changes in their physiology.

A few studies that outline some applications of HRV benefiting from real-time feedback include, but are not limited to clinical applications with real time relevance such as anticipation of mood changes in patients with Bipolar Disorder, alerting the onset of infant physiological dysfunction during sleep, early warning of epileptic seizure, food allergy alerting, and sleep apnea; and non-clinical applications with real time relevance such as predicting the onset of lactate threshold in endurance athletes, warning of physiological effects of pollution, particularly volatile organic compounds (VOCs), alerting the onset of driver fatigue, and monitoring professionals in high stress occupations (e.g., air traffic controllers, stock market traders). These scenarios and many others may benefit from the accurate monitoring, analysis and real time alerting, to a relevant change in physiology as indicated by a change in HRV.

Devices and methods for carrying out the invention are presented in terms of embodiments depicted within the FIGS. However, the invention is not limited to the described embodiments, and a person skilled in the art will appreciate that many other embodiments of the invention are possible without deviating from the basic concept of the invention, and that any such work around will also fall under scope of this invention. It is envisioned that other styles and configurations of the present invention can be easily incorporated into the teachings of the present invention, and the configurations shall be shown and described for purposes of clarity and disclosure and not by way of limitation of scope.

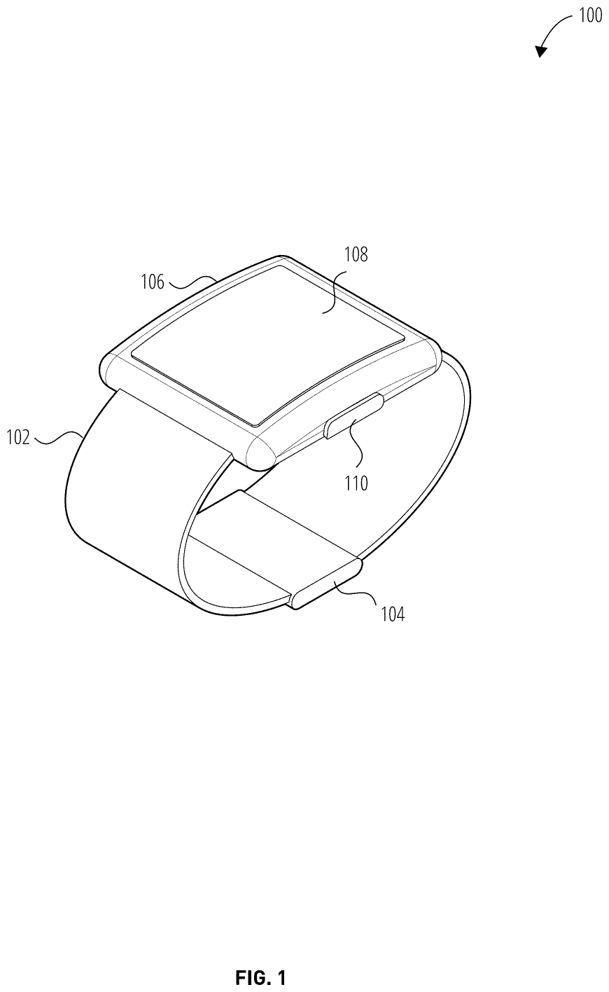

FIG. 1 shows a perspective view of an embodiment of the invention. A housing 106 is attached to a band 102. The band 102 is connected by means of a clasp 104. In the embodiment of the invention as shown, the housing 106 also comprises a screen 108 and a button 110. The button activates different modes or settings and can be used to acknowledge receipt of a signal.

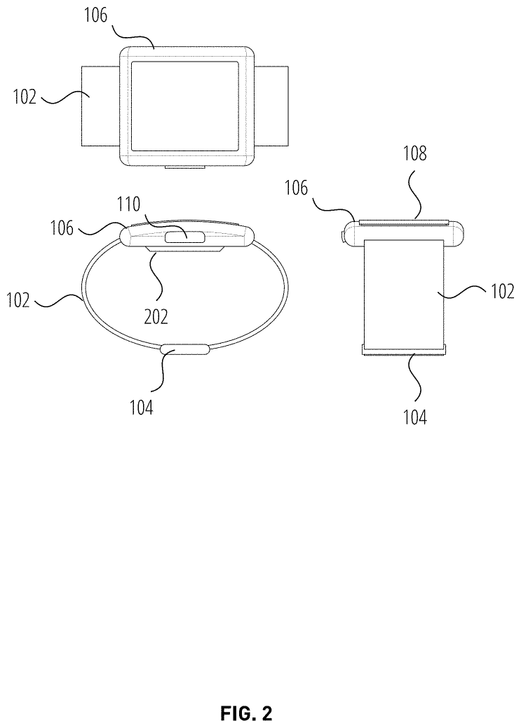

FIG. 2 shows top and side views of different embodiments of the invention. This figure shows the relative positions of the band 102, clasp 104, housing 106, screen 108 and button 110. In addition, the position of the sensor 202 can be seen on the underside of the housing 106. The sensor 202 detects and stores information about the users heart rate.

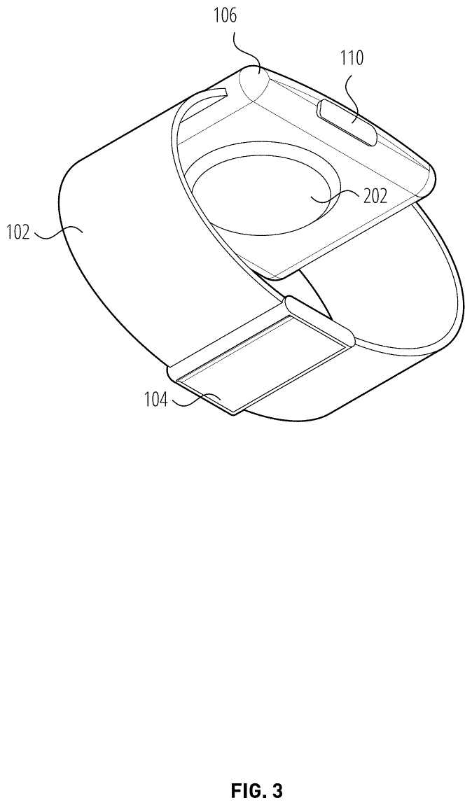

FIG. 3 shows a perspective view of an embodiment of the invention. This figure shows the relative positions of the band 102, clasp 104, housing 106, screen 108 and button 110. Again, the position of the sensor 202 can be seen on the underside of the housing 106. The sensor 202 has photodiode sensors and green and infrared LEDs. By flashing the LED lights hundreds of times per second, the device measures and calculates the number of times the heart beats each minute, thereby providing the heart rate (HR).

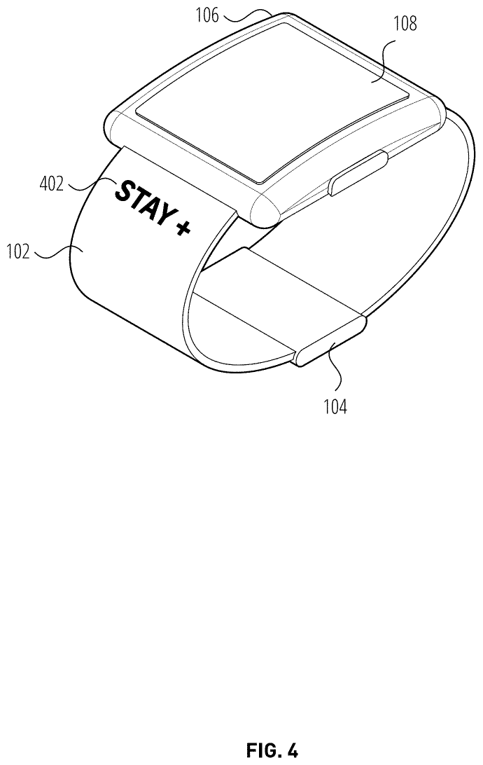

FIG. 4 shows a perspective view of an embodiment of the invention. This figure shows the relative placement of a company logo 402 on the band 102.

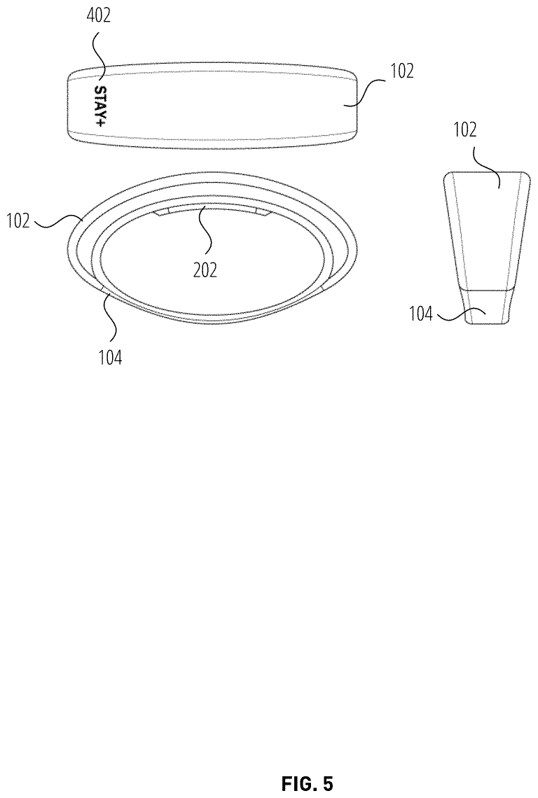

FIG. 5 shows side and top views of an alternative embodiment of the invention lacking a screen 108. The housing 106 is not visible as it is integral within the band 102. The sensor 202 is again located on the underside of the band 102 such that it can sense and record the user's heartbeat. The band 102 may be secured in place on the user's wrist by a clasp 104, or the band 102 may be fabricated from a material, such as silicone, that has flexibility and extensibility properties.

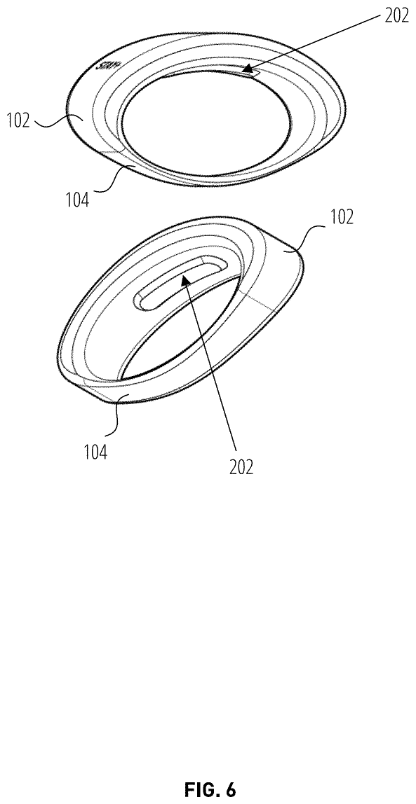

FIG. 6 shows a perspective view of the alternative embodiment of the invention lacking a screen 108. The relative positions of the band 102, clasp 104 and sensor 202 can be seen. Again, the sensor 202 is located on the underside of the band 102 such that it can sense and record the user's heartbeat.

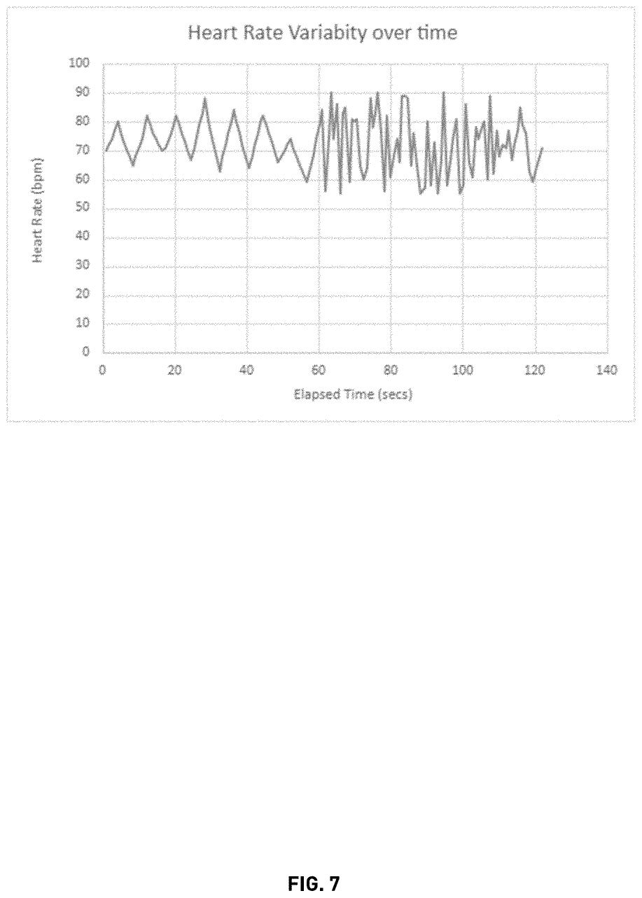

FIG. 7 shows the results of heart rate variability over the course of a 2 min time period as measured by an embodiment of the device. The first 60 seconds show a heart rate that is varying up and down and the COD is within an acceptable range (and thus no warning/notification at the end of that one minute). However, from 60-120 secs the heart rate is varying up and down at a very rapid rate and the COD is not within an acceptable range, hence a notification would be sent to the wearer at the second minute.

Based on a unique programmed formula determine and warn the wearer of the device that he/she is falling into an undesirable mental state (subconsciously/consciously), including a state of mental negativity.

The device measures and records the heart rate of the wearer. The microprocessor records if the trend of the heart rate is increasing, decreasing or remaining the same over a predetermined period of time. The programmed formula determines the elapsed time between each change of direction (COD) of the heart rate. The program calculates ranges of CODs that are considered acceptable, and provides notification to the wearer if and when the ranges of COD's that are considered problematic.

Specifically, in one embodiment of the invention, the formula looks to count the elapsed time per COD and counts the close proximity of each COD in terms of elapsed time and if that proximity is with 10% of the previous cycle it is counted as a successful/good COD. At the end of every minute a subsequent calculation is performed to calculate the percentage of successful/good CODs to the overall total CODs in that minute. The user will receive no warning at all if the score/percentage is higher or equal to 75%. If the score is less than 25% the customer will receive a more intense warning (vibration/noise), and medium to light warning if the score is less than 50% or 75% respectively.

The device is programmed to recognize the wearer's normal variation on heart rate and only notify the wearer when potentially problematic variates are encountered. For example, a person's heart rate normally varies, but the way in which it is varying illustrates the person's condition. If the wearer transitions from resting to running, the wearer's heart rate will increase quickly at first and then stabilize at a higher rate than when the wearer was resting. It will then exhibit a normal type of variation around this elevated level. When the wearer stops running, the wearer's heart rate will begin to decline until it reaches the resting state. The initial increase in heart rate will be faster than the subsequent decrease. It is not the fact that there are increases and decreases in heart rate that is cause for concern—it is the pattern of those increases and decreases. Regular or predictable patterns are not problematic, it is when the pattern becomes irregular that is a cause for concern as such changes can indicate a physiological reaction to an disturbing stimulus—a so-called “Amygdala Hijack”, when the most basic part of the brain, the amygdala, stimulates a “fight or flight” type reaction in a person. The heart rate will suddenly change but when the logic centers of the brain process the data and determine that there is no threat, the person will begin to relax. But it is not only in life-threatening situations that such reactions can occur. If the wearer is in a meeting where they do not like one of the participants and their contributions annoy them (or even just their presence), they can have a low-grade response to the person and trigger irregular changes in their heart rate variation. Being made aware that this is happening allows an individual to temper their response and to ensure that they give due consideration to the person's contribution before they respond.

Table 1 shows the results of heart rate variability over the course of a 2 min time period as measured by an embodiment of the device. The first 60 seconds show a heart rate that is varying up and down and the COD is within an acceptable range (and thus no warning/notification at the end of that one minute). However, from 60-120 secs the heart rate is varying up and down at a very rapid rate and the COD is not within an acceptable range, hence a notification would be sent to the wearer at the second minute.

The irregular change to the heart rate variation is noted by measuring the rate of change of heart rate variation. When a person is at rest, the variation in their heart rate might be in the region of +/−two beats per minute between successive beats. This a person at rest might record 60, 61, 63, 62, 61, 59, 60, 62, 62, 61, 60 and 59 as their heart rate as calculated in beats per minute (bpm) over a series of twelve beats. Their heart rate varies by one or two bpm per beat. If that person starts to exercise, their record might be 60, 75, 88, 100, 110, 107, 112, 115, 117, 118, 119, 120. In this situation, the initial increase of 15 bpm was unexpected, but the subsequent pattern is not. The system records one irregular change followed 11 reasonable changes, so there is no problematic variation in heart rate. However, a person in a stressful situation might record 60, 75, 78, 75, 68, 63, 78, 77, 68, 65, 79, 80 in response to a situation where they are receiving emotional shocks to their system. In this case, multiple changes in their heart rate reflect irregular variation and the person may benefit from assistance to calm their thoughts and emotions. Depending on the programming of the device, notifications of varying strength will be sent to the wearer to alert them to the fact that they need to employ whatever calming, relaxing, or meditative remedies known to them in order to temper their physiological reaction as indicated by the problematic heart rate variability.

In different embodiments of the invention, the device may be programmed for wearer's who have health concerns that are clinically indicated by either an increase or decrease in HRV. For example, a prolonged decrease in HRV may be related to many disorders including hypertension, arrhythmia, myocardial infarction, chronic fatigue, panic attacks and stress related disorders. It is generally accepted that a higher HRV has been found to be associated with reduced morbidity and mortality, and improved psychological well-being and quality of life.

Moreover, tracking instantaneous HRV may be a great tool to motivate behavioral change for some. HRV measurements can help create more awareness of how you live and think, and how your behavior affects your nervous system and bodily functions. While it obviously can't help you avoid stress, it could help you understand how to respond to stress in a healthier way.

The foregoing descriptions of specific embodiments of the present invention have been presented for purposes of illustration and description. They are not intended to be exhaustive or to limit the invention and method of use to the precise forms disclosed. Many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient, but is intended to cover the application or implementation without departing from the spirit or scope of the claims of the present invention.

1. A heart rate variability notification device for a user, the device comprising:
an output system configured to transmit a notification signal perceptible by the user based on the output system receiving an activation signal;
a plurality of sensors configured to produce electrical signals based on electrical activity of the user's heart;
a housing sized and configured to be worn by the user;
a memory housed by the housing wherein the memory is configured to store heart rate variability notification criteria; and
a processor housed by the housing and coupled to the plurality of sensors and the output system and the memory, the processor configured to receive digital signals associated with the electrical signals produced by the sensors, the processor configured to measure and record a measured heart rate variability of the user based on the digital signals and to transmit the activation signal to the output device based on whether the measured heart rate variability satisfies the heart rate variability feedback criteria, the processor configured to modify the heart rate variability feedback criteria based on the measured and recorded heart rate variability.

2. The heart rate variability notification device of claim 1, wherein the memory is further configured to store a goal threshold associated with the heart rate variability feedback criteria and wherein the processor is further configured to adjust a median threshold toward the goal threshold if the measured heart rate variability satisfies the median threshold and to transmit the activation signal to activate the output device if the measured heart rate variability does not satisfy the median threshold.

3. The heart rate variability notification feedback system of claim 1, wherein the memory is further configured to store heart rate variability data as stored heart rate variability data and wherein the processor is further configured to determine a trend based on the stored heart rate variability data and to modify the heart rate variability feedback criteria base on the trend.

4. The heart rate variability notification device of claim 1, wherein the memory is further configured to store previously measured heart rate variability as stored heart rate variability data and wherein the processor is further configured to determine a trend based on the stored heart rate variability data and the measured heart rate variability and to adjust the activation signal to subsequently adjust the notification signal based on the trend.

5. The heart rate variability notification device of claim 1, wherein the heart rate variability feedback criteria and determined heart rate variability are associated with time domain or frequency domain calculations associated with the electrical signals produced by the electrodes.

6. The heart rate variability notification device of claim 4, wherein the housing comprises a band and clasp configured to be worn around a wrist of the user.

7. The heart rate variability notification device of claim 4, wherein the housing additionally comprises a screen.

8. The heart rate variability notification device of claim 6, wherein the notification signal is selected from the group consisting of a haptic signal, an auditory signal or a visual signal.

9. The heart rate variability notification device of claim 6, wherein the haptic signal is a vibration.

10. The heart rate variability notification device of claim 7, wherein the visual signal is displayed on the screen.

11. The heart rate variability notification device of claim 10, wherein the visual signal consists of an image, message, inspirational quote, of GIF.

12. A method of alerting a wearer to problematic variations in their heart rate, said method comprising the wearer conducting the following steps:
affixing the device of claim 1, onto a wrist of the wearer;
receiving a notification from said device warning that problematic variations in the wearer's heart rate have been encountered; and
modulating the behavior to use calming, relaxing, or meditative remedies known to them in order to temper their physiological reaction as indicated by the problematic variations in the wearer's heart rate variability.
